(19) United States
(12) Patent Application Publication
(10) Pub. No.: US 2023/0091992 A1
(43) Pub. Date: Mar. 23, 2023

(54) Pelvic-Based Alignment System

(71) Applicant: Dill-Davidson Innovations, LLC, Davidson, NC (US)

(72) Inventors: Lisa Davidson, Stanley, NC (US); Scott Davidson, Sunnyvalye, CA (US); Dylan Davidson, Sunnyvale, CA (US); Shmuel Silverman, Novato, CA (US)

(73) Assignee: Dill-Davidson Innovations, LLC, Davidson, NC (US)

(21) Appl. No.: 17/942,113

(22) Filed: Sep. 10, 2022

Related U.S. Application Data
(60) Provisional application No. 63/244,635, filed on Sep. 15, 2021.

(51) Int. Cl. A61B 5/00; A61B 5/11

(52) U.S. Cl. CPC A61B 5/486; A61B 5/6823; A61B 5/6831; A61B 5/1116; A61B 2562/0219

(57) ABSTRACT

An alignment system that monitors posture and provides alignment feedback about the monitored posture to an individual is described. This alignment system may include an alignment device that is worn or remateably attached to the body of the individual, e.g., in or near a midline (such as at or near a midpoint between the Iliac Fossa) of the posterior lumbar or sacral region. The alignment device may include one or more alignment sensors and/or may communicate with one or more separate alignment sensors that monitor the alignment of the individual's pelvis. This alignment data may be analyzed by the alignment device or another electronic device (such as an application executed on the other electronic device and/or a remotely located computer). Then, based at least in part on the analysis, the alignment device may selectively provide the alignment feedback to the individual (e.g., when misalignment is detected or determined).

### CROSS REFERENCE TO RELATED APPLICATIONS

This application claims priority under 35 U.S.C. 119(e) to U.S. Provisional Application Ser. No. 63/244,635, “Pelvic-Based Alignment System,” filed on Sep. 15, 2021, by Lisa Davidson, et al., the contents of which are herein incorporated by reference.

### FIELD

The disclosed embodiments relate to feedback techniques that provide alignment feedback about posture of an individual. Notably, the disclosed embodiments relate to an alignment system that monitors the posture of an individual and that provides alignment feedback about the monitored posture to the individual.

### BACKGROUND

The Centers for Disease Control and Prevention estimated 50 million adults in the U.S. have experienced chronic pain, with 19.6 million experiencing high-impact chronic pain. This equates to roughly 1 in 5 adults, with most noting pain in the lower back, neck, and/or headaches and migraines. Chronic pain costs the U.S. up to $635 billion dollars every year.

Often, chronic pain is associated with (if not caused by) problems with body mechanics, such as kinesthesia or posture errors. However, it is often difficult for people to appreciate the impact that body mechanics has on their well-being or to notice when their body mechanics (such as their alignment) is skewed or in error.

Given the number of people afflicted with chronic pain, it is not surprising that many patients seek out professional assistance from a variety of healthcare professions. While chiropractors and physical therapists can provide feedback and can assist patients in improving their alignment and posture, appointments with such healthcare professionals are at best episodic and often are infrequent. Consequently, it is difficult for individuals to receive regular or continuous feedback about the alignment and posture errors, which makes it difficult for these individuals to learn better habits and to carry their bodies in ways that can reduce (or even eliminate) chronic pain.

### SUMMARY

An alignment device is described. This alignment device includes an integrated circuit (such as a processor) that: obtains measurements associated with a pelvic posture of an individual; analyzes the measurements to determine when a posture misalignment occurs; and when the posture misalignment occurs, provides feedback intended for the individual, where the alignment device is remateably attached or coupled to the individual.

Note that the alignment device may be remateably attached or coupled to the individual near a midline of the posterior lumbar or sacral region.

Moreover, the alignment device may include one or more alignment sensors that perform the measurements, where obtaining the measurements may include acquiring the measurements using the one or more alignment sensors. Furthermore, the one or more sensors may include: an orientation sensor, a gyroscope, a compass and/or an accelerometer.

Alternatively or additionally, the alignment device may include an interface circuit that communicates with one or more second alignment sensors that are external to the alignment device, where obtaining the measurements may include acquiring the measurements using the one or more alignment sensors.

In some embodiments, the alignment device may include an interface circuit that communicates with an electronic device, where analyzing the measurements may include: providing, addressed to the electronic device, the measurements; and receiving, associated with the electronic device, results of the analysis and/or instructions for the feedback.

Note that the feedback may include: an indication that the posture misalignment has occurred; or a recommended therapy to correct the posture misalignment. Moreover, the feedback may include a sensory output intended for the individual. Furthermore, the feedback may include: light, sound, and/or haptic information.

Additionally, the posture misalignment may include a pelvic misalignment.

Another embodiment provides an electronic device. This electronic device may include: an interface circuit that communicates with an alignment device; a processor; and memory that stores program instructions, where, when executed by the processor, the program instructions cause the electronic device to perform operations including: receiving, associated with the alignment device, measurements associated with a pelvic posture of an individual; analyzing the measurements to determine when a posture misalignment occurs; and when the posture misalignment occurs, providing, addressed to the alignment device, feedback intended for the individual.

Alternatively, the electronic device may include: an image sensor that acquires images; a processor; and memory that stores program instructions, where, when executed by the processor, the program instructions cause the electronic device to perform operations including: acquiring, using the image sensor, one or more images associated with a posture of an individual; analyzing the one or more images to determine when a posture misalignment occurs; and when the posture misalignment occurs, providing feedback intended for the individual.

Another embodiment provides a computer system. This computer system includes: an interface circuit that communicates with an electronic device; a processor; and memory that stores program instructions, where, when executed by the processor, the program instructions cause the computer system to perform operations including: receiving, associated with the electronic device, measurements associated with a pelvic posture of an individual; analyzing the measurements to determine when a posture misalignment occurs; and when the posture misalignment occurs, providing, addressed to the electronic device, feedback intended for the individual.

Another embodiment provides a computer-readable storage medium for use with the alignment device, an embodiment of the electronic device or a computer system. This computer-readable storage medium may include program instructions that, when executed by the alignment device, the electronic device or the computer system, cause the alignment device, the electronic device or the computer system to perform at least some of the aforementioned operations.

Another embodiment provides a method. This method includes at least some of the operations performed by the alignment device, the electronic device or the computer system.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a drawing illustrating an example of communication between electronic devices in accordance with an embodiment of the present disclosure.

FIG. 2 is a block diagram illustrating an example of an alignment device in accordance with an embodiment of the present disclosure.

FIG. 3 is a flow diagram illustrating an example method for providing feedback using an electronic device in FIG. 1 in accordance with an embodiment of the present disclosure.

FIG. 4 is a flow diagram illustrating an example method for providing feedback using an electronic device in FIG. 1 in accordance with an embodiment of the present disclosure.

FIG. 5 is a flow diagram illustrating an example method for providing feedback using an electronic device in FIG. 1 in accordance with an embodiment of the present disclosure.

FIG. 6 is a drawing illustrating an example of communication between electronic devices in FIG. 1 in accordance with an embodiment of the present disclosure.

FIG. 7 is a drawing illustrating an example comparing posture misalignment with posture alignment in accordance with an embodiment of the present disclosure.

FIG. 8 is a drawing illustrating an example of positioning of an alignment device in FIG. 1 in accordance with an embodiment of the present disclosure.

FIG. 9 is a drawing illustrating an example of adhesive-based attachment of an alignment device in FIG. 1 in accordance with an embodiment of the present disclosure.

FIG. 10 is a drawing illustrating an example of belt-based attachment of an alignment device in FIG. 1 in accordance with an embodiment of the present disclosure.

FIG. 11 is a drawing illustrating an example of clothing-based attachment of an alignment device in FIG. 1 in accordance with an embodiment of the present disclosure.

FIG. 12 is a drawing illustrating an example of scapula-based attachment of an alignment device in FIG. 1 in accordance with an embodiment of the present disclosure.

FIG. 13 is a drawing illustrating an example of posture monitoring using an electronic device that includes an image sensor in accordance with an embodiment of the present disclosure.

FIG. 14 is a drawing illustrating an example of anatomical planes of the human body in accordance with an embodiment of the present disclosure.

FIG. 15 is a drawing illustrating an example of a mesh network in accordance with an embodiment of the present disclosure.

FIG. 16 is a drawing illustrating an example of an alignment-system feedback structure in accordance with an embodiment of the present disclosure.

FIG. 17 is a drawing illustrating an example of an alignment-device interaction in accordance with an embodiment of the present disclosure.

FIG. 18 is a drawing illustrating an example of communication between electronic devices in FIG. 1 in accordance with an embodiment of the present disclosure.

FIG. 19 is a drawing illustrating an example of communication between electronic devices in FIG. 1 in accordance with an embodiment of the present disclosure.

FIG. 20 is a flow diagram illustrating an example method for alignment triggering using an electronic device in FIG. 1 in accordance with an embodiment of the present disclosure.

FIG. 21 is a drawing illustrating an example of sampled measurements in accordance with an embodiment of the present disclosure.

FIG. 22 is a drawing illustrating an example of proximate measurements in accordance with an embodiment of the present disclosure.

FIG. 23 is a drawing illustrating an example of communication between electronic devices in FIG. 1 in accordance with an embodiment of the present disclosure.

FIG. 24 is a block diagram illustrating an example of an electronic device in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

An alignment system that monitors posture of an individual and that provides alignment feedback about the monitored posture to the individual is described. This alignment system may include an alignment device that is worn or remateably attached to (or mounted to) the body of the individual, e.g., in or near a midline (such as at or near a midpoint between the Iliac Fossa) of the posterior lumbar or sacral region. The alignment device may include one or more alignment sensors and/or may communicate with one or more separate alignment sensors that monitor the alignment of the individual's pelvis. This alignment data may be analyzed by the alignment device or another electronic device (such as an application executed on the other electronic device, e.g., a cellular telephone and/or a remotely located computer, e.g., a cloud-based server). Then, based at least in part on the analysis, the alignment device may selectively provide the alignment feedback to the individual (e.g., when misalignment is detected or determined).

By selectively providing the feedback, the feedback techniques may improve awareness by the individual about occurrences of posture misalignment. Moreover, the feedback may provide timely (e.g., real time) guidance or recommendations to the individual about how they can correct posture misalignment. Thus, the feedback techniques may promote improved posture and posture alignment, and may reduce pain and other sequelae associated with posture misalignment. Consequently, the feedback techniques may promote the well-being of the individual.

In the discussion that follows, electronic devices or components in a system communicate packets in accordance with a wireless communication protocol, such as: a wireless communication protocol that is compatible with an IEEE 802.11 standard (which is sometimes referred to as ‘Wi-Fi®,’ from the Wi-Fi Alliance of Austin, Tex.), Bluetooth (such as Bluetooth classic or Bluetooth low energy or BLE), a cellular-telephone network or data network communication protocol (such as a third generation or 3G communication protocol, a fourth generation or 4G communication protocol, e.g., Long Term Evolution or LTE (from the 3rd Generation Partnership Project of Sophia Antipolis, Valbonne, France), LTE Advanced or LTE-A, a fifth generation or 5G communication protocol, or other present or future developed advanced cellular communication protocol), and/or another type of wireless interface (such as another wireless-local-area-network interface). For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies. Moreover, an access point, a radio node, a base station or a switch in the wireless network may communicate with a local or remotely located computer (such as a controller) using a wired communication protocol, such as a wired communication protocol that is compatible with an IEEE 802.3 standard (which is sometimes referred to as ‘Ethernet’), e.g., an Ethernet II standard. However, a wide variety of communication protocols may be used in the system, including wired and/or wireless communication. In the discussion that follows, Wi-Fi, BLE and Ethernet are used as illustrative examples.

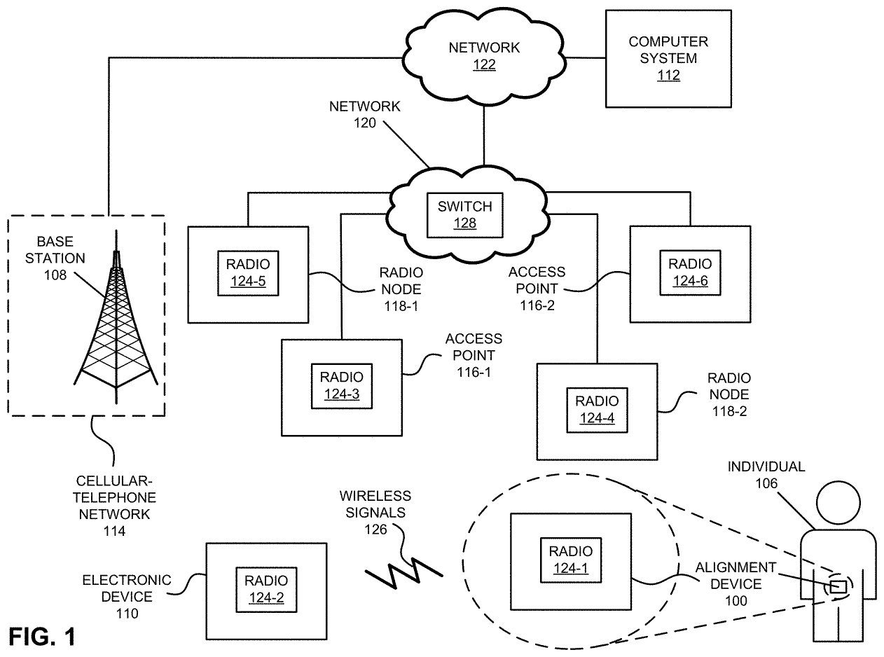

We now describe some embodiments of the feedback techniques. FIG. 1 presents a block diagram illustrating an example of communication in an environment with an electronic device 110 (such as a cellular telephone, a portable electronic device, a station or a client, another type of electronic device, etc.) via a cellular-telephone network 114 (which may include a base station 108), one or more access points 116 (which may communicate using Wi-Fi) in a WLAN and/or one or more radio nodes 118 (which may communicate using LTE) in a small-scale network (such as a small cell). For example, the one or more radio nodes 118 may include: an Evolved Node B (eNodeB), a Universal Mobile Telecommunications System (UMTS) NodeB and radio network controller (RNC), a New Radio (NR) gNB or gNodeB (which communicates with a network with a cellular-telephone communication protocol that is other than LTE), etc. In the discussion that follows, an access point, a radio node or a base station are sometimes referred to generically as a ‘communication device.’ Moreover, one or more base stations (such as base station 108), access points 116, and/or radio nodes 118 may be included in one or more wireless networks, such as: a WLAN, a small cell, and/or a cellular-telephone network. In some embodiments, access points 116 may include a physical access point and/or a virtual access point that is implemented in software in an environment of an electronic device or a computer. Furthermore, electronic device 110 may communicate with alignment device 100 using BLE.

Note that access points 116 and/or radio nodes 118 may communicate with each other and/or computer system 112 (which may be a local or a cloud-based computer system that includes one or more computers and that provides cloud-based storage and/or analytical services) using a wired communication protocol (such as Ethernet) via network 120 and/or 122. Note that networks 120 and 122 may be the same or different networks. For example, networks 120 and/or 122 may an LAN, an intra-net or the Internet. In some embodiments, network 120 may include one or more routers and/or switches (such as switch 128).

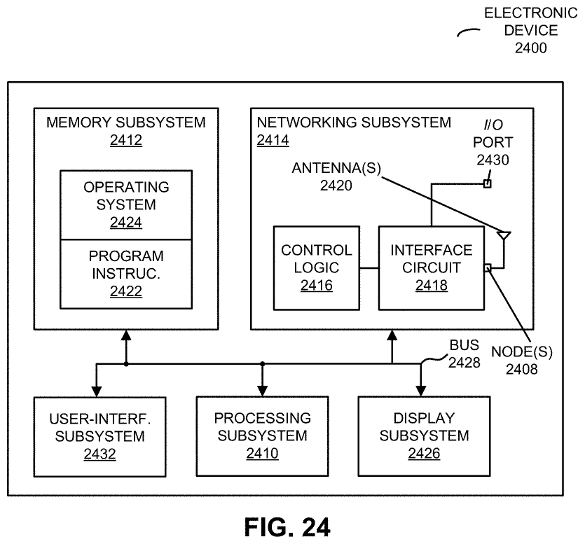

As described further below with reference to FIG. 24, alignment device 100, electronic device 110, computer system 112, access points 116, radio nodes 118 and switch 128 may include subsystems, such as a networking subsystem, a memory subsystem and a processor subsystem. In addition, alignment device 100, electronic device 110, access points 116 and radio nodes 118 may include radios 124 in the networking subsystems. More generally, alignment device 100, electronic device 110, access points 116 and radio nodes 118 can include (or can be included within) any electronic devices with the networking subsystems that enable alignment device 100, electronic device 110, access points 116 and radio nodes 118 to wirelessly communicate with one or more other electronic devices. This wireless communication can comprise transmitting access on wireless channels to enable electronic devices to make initial contact with or detect each other, followed by exchanging subsequent data/management frames (such as connection requests and responses) to establish a connection, configure security options, transmit and receive frames or packets via the connection, etc.

During the communication in FIG. 1, alignment device 100, electronic device 110, access points 116 and/or radio nodes 118 may wired or wirelessly communicate while: transmitting access requests and receiving access responses on wireless channels, detecting one another by scanning wireless channels, establishing connections (for example, by transmitting connection requests and receiving connection responses), and/or transmitting and receiving frames or packets (which may include information as payloads).

As can be seen in FIG. 1, wireless signals 126 (represented by a jagged line) may be transmitted by radios 124 in, e.g., alignment device 100 and electronic device 110. For example, radio 124-1 in alignment device 100 may transmit information (such as one or more packets or frames) using wireless signals 126. These wireless signals are received by radios 124 in one or more other electronic devices (such as radio 124-2 in electronic device 110). This may allow alignment device 100 to communicate information to electronic device 110. Note that wireless signals 126 may convey one or more packets or frames.

In the described embodiments, processing a packet or a frame in alignment device 100, electronic device 110, access points 116 and/or radio nodes 118 may include: receiving the wireless signals with the packet or the frame; decoding/extracting the packet or the frame from the received wireless signals to acquire the packet or the frame; and processing the packet or the frame to determine information contained in the payload of the packet or the frame.

Note that the wireless communication in FIG. 1 may be characterized by a variety of performance metrics, such as: a data rate for successful communication (which is sometimes referred to as ‘throughput’), an error rate (such as a retry or resend rate), a mean-squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). While instances of radios 124 are shown in components in FIG. 1, one or more of these instances may be different from the other instances of radios 124.

In some embodiments, wireless communication between components in FIG. 1 uses one or more bands of frequencies, such as: 900 MHz, 2.4 GHz, 5 GHz, 6 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. Note that the communication between electronic devices may use multi-user transmission (such as orthogonal frequency division multiple access or OFDMA) and/or multiple-input multiple-output (MIMO).

Although we describe the network environment shown in FIG. 1 as an example, in alternative embodiments, different numbers or types of electronic devices may be present. For example, some embodiments comprise more or fewer electronic devices. As another example, in another embodiment, different electronic devices are transmitting and/or receiving packets or frames.

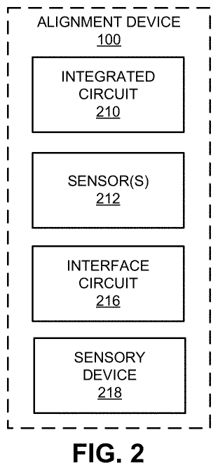

As discussed previously, it can be difficult for an individual to obtain feedback about their body mechanics, such as their posture. Moreover, as described further below with reference to FIGS. 2-23, in order to addresses these problems alignment device 100 may be used to determine when a posture misalignment (such as pelvic misalignment) occurs. Notably, alignment device 100 may be remateably attached or coupled to an individual 106 near a midline of the posterior lumbar or sacral region. For example, alignment device 100 may be: attached to individual 106 using an adhesive; attached to a belt and worn by individual 106; or attached to an article of clothing that is worn by individual 106.

Moreover, alignment device 100 may include one or more alignment sensors that perform measurements associated with a pelvic posture of an individual. For example, the one or more sensors may include: an orientation sensor, a gyroscope, a compass and/or an accelerometer. Alternatively or additionally, the alignment device 100 may include an interface circuit that communicates with one or more second alignment sensors that are external to alignment device 100, and alignment device 100 may obtain the measurements using the one or more alignment sensors.

Then, alignment device 100 may analyze the measurements to determine when the posture misalignment occurs. In some embodiments, alignment device 100 provides the measurements to electronic device 110, which performs at least a portion of the analysis, and then provides results of the analysis and/or instructions for feedback. Alternatively or additionally, the measurements are provided by alignment device 100, directly or indirectly (e.g., via electronic device 110), to computer system 112, which performs at least a portion of the analysis, and then provides the results of the analysis and/or instructions for the feedback. Thus, in some embodiments, the analysis is performed solely by alignment device 100, solely by electronic device 110, solely by computer system 112, or jointly by alignment device 100, electronic device 110 and/or computer system 112.

Next, alignment device 100 provides the feedback to individual 106. For example, the feedback may include: an indication that the posture misalignment has occurred; or a recommended therapy to correct the posture misalignment. Moreover, the feedback may include a sensory output intended for individual 106, such as: light, sound, and/or haptic information. In some embodiments, the feedback may include a verbal instruction or command that is provided by alignment device 100, e.g., using a voice-synthesis engine (such as a pretrained neural network). In some embodiments, alignment device 100 may determine the feedback dynamically, e.g., based at least in part on the occurrence of the posture misalignment. However, in some embodiments, alignment device 100 may select the feedback from a set of predetermined feedback, e.g., based at least in part on the occurrence of the posture misalignment.

In some embodiments, the sensitivity or configuration of alignment device 100 to perform feedback (such as haptic feedback) may be dynamically adapted or changed, e.g., as part of the recommended therapy. For example, when an individual is doing better (such as when their posture misalignment is improved), a haptic threshold range may be reduced. Alternatively, for a new user, the haptic threshold range may be increased. Thus, the feedback may include a change to the configuration of the haptic response provided by alignment device 100. Note that the haptic threshold range may be dynamically adapted using a pretrained predictive model, such as a machine-learning model or a neural network. Additionally, in some embodiments, the haptic threshold range may be based at least in part on a daily and/or weekly schedule of an individual. Consequently, the haptic threshold range may be different during standing/sitting during work hours that a time when the individual is running, lifting weights, riding a bicycle, etc.

While the preceding discussion illustrated the feedback techniques with alignment device 100 remateably coupled to or being located on or proximate to individual 106, in other embodiments the measurements are performed at a distance from individual 106 and occurrence of posture misalignment is inferred from these measurements. In these embodiments, alignment device 100 may not be needed. Notably, in some embodiments, electronic device 110 may include an image sensor (such as a CCD sensor, a CMOS sensor and, more generally, a camera). Using the image sensor, electronic device 110 may acquire one or more images associated with a posture of individual 106. Then, electronic device 110 may analyze the one or more images to determine when the posture misalignment occurs. (As in the preceding embodiments, this analysis may be performed solely by electronic device 110, may be performed by computer system 112 or may be performed jointly by electronic device 110 and computer system 112. Moreover, when the posture misalignment occurs, electronic device 110 may provide feedback intended for individual 106.

In these ways, the feedback techniques may improve understanding of individual 106 about occurrences of posture misalignment, and may provide timely (e.g., real time) guidance or recommendations to individual 106 about how they can correct posture misalignment.

FIG. 2 presents a block diagram illustrating an example of alignment device 100. This alignment device may include an integrated circuit 210 that performs at least some of the operations in the feedback techniques. For example, integrated circuit 210 may include a processor that executes program instructions or software that cause alignment device 100 to perform at least some of the operations in the feedback techniques.

In some embodiments, alignment device 100 may optionally include one or more sensors 212 that perform at least some of the measurements. Alternatively or additionally, alignment device 100 may optionally include an interface circuit 214 that communicates with one or more external sensors that perform at least some of the measurements.

Moreover, in some embodiments, interface circuit 214 may communicate with electronic device 110 (FIG. 1). This may facilitate exchange of the measurements, analysis of the measurements and/or determining or selecting of the feedback.

Furthermore, alignment device 100 may include a sensory device 216, such as: an LED, a speaker, a voice coil that generates vibrations and/or a display. This sensory device may allow alignment device 100 to provide the feedback to the individual.

In some embodiments, alignment device 100 may include fewer or more components, two or more components may be combined into a single component, a second single component may be implemented using multiple components, and/or a position of at least one of the components may be changed.

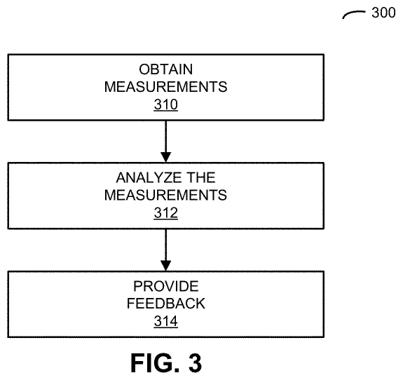

We now describe embodiments of the method. FIG. 3 presents a flow diagram illustrating an example of a method 300 for providing feedback using an electronic device, such as alignment device 100 (FIG. 1). During operation, the alignment device may obtain measurements (operation 310) associated with a pelvic posture of an individual. Then, the alignment device may analyze the measurements (operation 312) to determine when a posture misalignment (such as a pelvic misalignment) occurs. When the posture misalignment occurs, the alignment device may provide the feedback (operation 314) intended for the individual, where the alignment device is remateably attached or coupled to the individual. Note that the alignment device may be remateably attached or coupled to the individual near a midline of the posterior lumbar or sacral region.

Moreover, the alignment device may include one or more alignment sensors that perform the measurements, where obtaining the measurements (operation 310) may include acquiring the measurements using the one or more alignment sensors. Note that the one or more sensors may include: an orientation sensor, a gyroscope, a compass and/or an accelerometer. Alternatively or additionally, the alignment device may include an interface circuit that communicates with one or more second alignment sensors that are external to the alignment device, where obtaining the measurements (operation 310) may include acquiring the measurements using the one or more alignment sensors. In some embodiments, the alignment device may include an interface circuit that communicates with an electronic device, where analyzing the measurements (operation 310) may include: providing, addressed to the electronic device, the measurements; and receiving, associated with the electronic device, results of the analysis and/or instructions for the feedback.

Note that the feedback may include: an indication that the posture misalignment has occurred; or a recommended therapy to correct the posture misalignment. Moreover, the feedback may include a sensory output intended for the individual. Note that the feedback may include: light, sound, and/or haptic information.

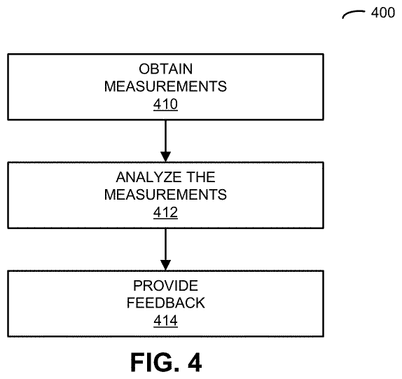

FIG. 4 presents a flow diagram illustrating an example of a method 400 for providing feedback using an electronic device, such as electronic device 110 (FIG. 1). During operation, the electronic device may obtain measurements (operation 410) associated with a pelvic posture of an individual. For example, the electronic device may receive, associated with an alignment device, measurements associated with a pelvic posture of an individual. Then, the electronic device may analyze the measurements (operation 412) to determine when a posture misalignment occurs. When the posture misalignment occurs, the electronic device may provide, addressed to the alignment device, the feedback (operation 414) intended for the individual.

Alternatively, in some embodiments, the electronic device may include an image sensor that acquires images, and the electronic device may obtain the measurements (operation 410) by acquiring, using the image sensor, one or more images associated with a posture of an individual, which may then be analyzed (operation 412) by the electronic device to determine when the posture misalignment occurs; and when the posture misalignment occurs.

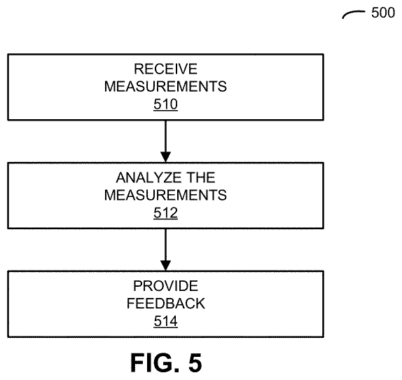

FIG. 5 presents a flow diagram illustrating an example of a method 500 for providing feedback using an electronic device, such as computer system 112 (FIG. 1). During operation, the computer system may receive, associated with an electronic device, measurements (operation 510) associated with a pelvic posture of an individual. Then, the computer system may analyze the measurements (operation 512) to determine when a posture misalignment occurs. When the posture misalignment occurs, the computer system may provide, addressed to the electronic device, the feedback (operation 514) intended for the individual.

In some embodiments, method 300 (FIG. 3), 400 (FIG. 4) and/or 500 may include additional or fewer operations. Moreover, the order of the operations may be changed, there may be different operations, two or more operations may be combined into a single operation, and/or a single operation may be divided into two or more operations.

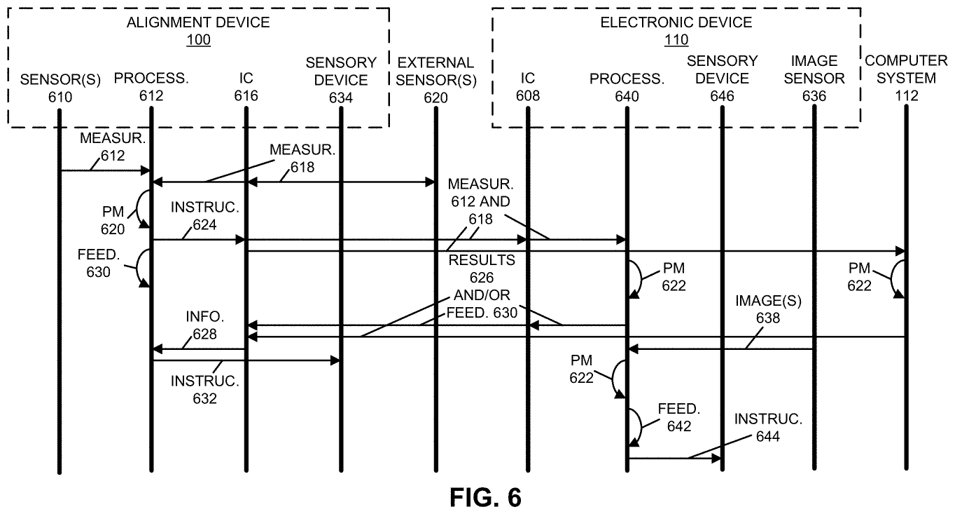

Embodiments of the feedback techniques are further illustrated in FIG. 6, which presents a drawing illustrating an example of communication between alignment device 100, electronic device 110 and computer system 112. Notably, one or more sensors 610 in alignment device 100 may perform measurements 612 associated with a pelvic posture of an individual. These measurements may be provided to processor 614 in alignment device 100. Alternatively or additionally, interface circuit (IC) 616 may acquirement measurements 618 associated with the pelvic posture of the individual from external sensors 620, which are then provided to processor 614.

Then, processor 614 may analyze measurements 612 and/or measurements 618 to determine when a posture misalignment (PM) 622 occurs. Moreover, when posture misalignment 622 occurs, processor 614 may determine or may select feedback 630.

Alternatively or additionally, processor 614 may instruct 624 interface circuit 616 to provide measurements 612 and/or measurements 618 to electronic device 110 and/or computer system 112. After receiving measurements 612 and/or measurements 618, interface circuit 608 in electronic device 110 may provide measurements 612 and/or measurements 618 to processor 640 in electronic device 110, which may analyze measurements 612 and/or measurements 618 to determine when posture misalignment 622 occurs, and may at least selectively provide results 626 of the analysis (such as when posture misalignment 622 occurs) and/or feedback 630 to alignment device 100. Similarly, computer system 112 may receive and then may analyze measurements 612 and/or measurements 618 to determine when posture misalignment 622 occurs, and may at least selectively provide results 626 of the analysis (such as when posture misalignment 622 occurs) and/or feedback 630 to alignment device 100. Moreover, after receiving results 626 and/or feedback 630, interface circuit 616 may provide information 628 indicating that posture misalignment 622 has occurred and/or feedback 630 to processor 614.

Furthermore, processor 614 may instruct 632 sensory device 634 in alignment device 100 to provide feedback 630 intended for the individual.

Alternatively, in some embodiments, electronic device 110 may include an image sensor 636. Using image sensor 636, electronic device 110 may acquire one or more images 638 associated with a posture of the individual, which are provided to a processor 640. Then, processor 640 may analyze the one or more images 638 to determine when posture misalignment 622 occurs. Moreover, when posture misalignment 622 occurs, processor 640 may determine or select feedback 642, and processor 640 may instruct 644 sensory device 646 in electronic device 110 to provide feedback 642 intended for the individual.

While FIG. 6 illustrates communication between components using unidirectional or bidirectional communication with lines having single arrows or double arrows, in general the communication in a given operation in this figure may involve unidirectional or bidirectional communication. Moreover, while FIG. 6 illustrates operations being performed sequentially or at different times, in other embodiments at least some of these operations may, at least in part, be performed concurrently or in parallel.

We now further describe the feedback techniques. The disclosed feedback techniques address an arbitrary type of misalignment in the human body. Notably, the feedback techniques address misalignment in the pelvis using an alignment device (which is sometimes referred to as an ‘alignment system’ or is a component in the alignment system) that helps determine where the pelvis is in relation to an individual's posture. The pelvis functions as the structural foundation of the body, and the remaining anatomical structures move in relation to the pelvis. For example, when an individual stands with an unbalanced amount of weight on each leg, the other side of their body has to overcompensate in an attempt to correct for this imbalance. This overcompensation can adversely impact other areas of the body, e.g., it may result or contribute to headache, sore back, sore feet, etc. By providing feedback that allows the pelvis to remain in a neutral position, the feedback techniques can alleviate other pains and problems in the body.

Posture theory asserts that misalignment in the body, and in particular in the pelvis, contributes to a large amount of discomfort throughout the body. For example, one type of posture imbalance or misalignment can occur when an individual repeatedly extends their hip to one side and puts more weight on the associated foot, thereby putting more body weight on the pelvic joint. Notably, this type of posture is commonly referred to as ‘hanging on one hip’ or ‘leaning on one leg.’

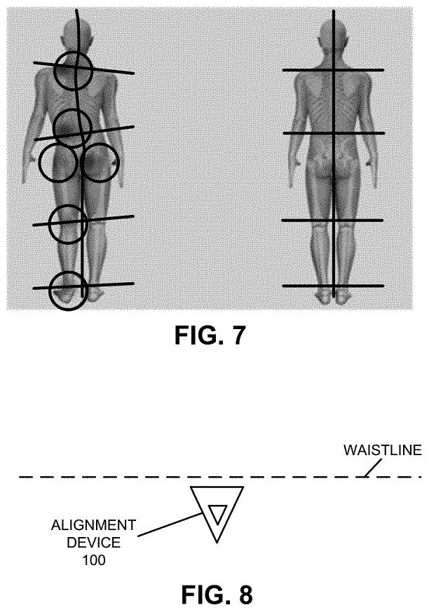

While little discomfort may be felt at the time, this constant misalignment can cause musculoskeletal issues throughout the body. For example, when an individual stands this way, they are putting excessive pressure on one side of their lower back and hip, affecting and potentially straining muscles and ligaments in the spine, hip, and neck. While the misalignment may seem only noticeable in the pelvic region, it can impact other parts of the body, e.g., radiating or moving from the pelvis, up the spine, and eventually to the neck. FIG. 7 presents a drawing illustrating an example comparing posture misalignment (and its impact on the body) with posture alignment.

Industry experts report that many clients they see are unaware that they have pelvic postural distortion. Whether the resulting pain is chronic or infrequent, those experiencing bodily pain thought to be in the bones, muscle, nervous system, or fascia, often seek relief from: chiropractic adjustments, acupuncture or acupressure, electro-stimulation therapy, massage therapy, and/or prescriptions. While this list is not exhaustive, it includes some of the common types of therapy individuals may seek to remedy pain or discomfort. Moreover, some people may try using physical devices to fix the pain at a specific location, but this often moves the pain to another area. For example, an individual may wear a neck support and the pain may move to their back because their pelvis is the core problem.

The discloses alignment system may address these problems. The alignment system may include at least three elements that can operate independently of each other and provide both direct feedback and derived analytics to the user following user setup and/or immediate requests. These three elements may include: an alignment device, an application installed on and executed on a separate electronic device (such as a cellular telephone, a computer or an Internet-of-things or IoT device), and a remote server or computer (such as a cloud-based computer or computer system) that is accessed via a network (such as the Internet or a cellular-telephone network). The alignment device may include one or more sensors (such as an orientation sensor, a gyroscope, a compass, an accelerometer, etc.) that monitor posture in real time (e.g., continuously, periodically or as-needed), and that optionally provide real-time feedback using one or more of: tactile pressure, light, sound, voice, vibration, etc. Note that the alignment device can be remateably physically attached or coupled to a user. Alternatively, the alignment device may use image sensors (or radar or LiDAR) and may be placed outside or away from the user. Furthermore, the application installed on and executed the separate electronic device may operate in one or more of multiple modes, including: communicating data with the remote server or computer, performing at least a portion of analysis of data, and/or providing feedback to the user (e.g., using visual, tactile pressure, light, sound, voice, vibration, etc.). Additionally, the remote server or computer may: collect long term information, perform some or all of the analysis of data, and/or provide cumulative or aggregated feedback and reports to the user.

The alignment device (which is sometimes referred to as a ‘posture device’) may be triangular in shape with tapered, rounded, or smooth edges. As shown in FIG. 8, which presents a drawing illustrating an example of positioning of an alignment device, the alignment device may be a cohesive or integrated device that includes multiple embedded components. Note that FIG. 8 provides an illustration of the general shape of the alignment device and how it may be worn. While not shown in FIG. 8, the alignment device may have tapered or rounded edges. In this example, the alignment device is located in the midline of the back, perfectly centered to the hip bones in the lower back, and may be positioned on the tailbone or coccyx.

It is possible for the alignment device to be attached to the body in several different ways and/or at several different locations. The different alignment-device attachment or mounting locations and various ways of attaching it to the body are described below.

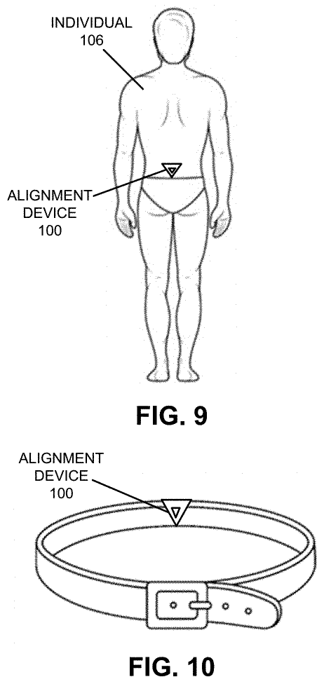

In some embodiments, the alignment device is remateably attached to the lower back using adhesives. The location of the alignment device may be below the waistline and positioned in or near the center of the back, just between the hip bones or Iliac bones. FIG. 9 presents a drawing illustrating an example of adhesive-based attachment of an alignment device.

This attachment technique may allow for easy attachment and may result in relatively accurate hip posture and overall pelvic-alignment measurements. If positioned correctly, the alignment device can take measurements of the hip displacement at any given time. While the sensor placements may be as shown above, in other embodiments the sensor placement may be based at least in part on physical activity.

For this type of alignment-device placement and attachment, the alignment or misalignment of the pelvis may be tracked, measured and shared with the separate electronic device, the remote server and/or software executed by the alignment device in order to provide actionable feedback to the user.

Alternatively, in some embodiments, the alignment device may be attached to a strap or belt that goes around the waist. This is shown in FIG. 10, which presents a drawing illustrating an example of belt-based attachment of an alignment device.

As shown in FIG. 10, the alignment device may be located on the belt. Notably, the alignment device may be placed in a location where it will be centered or nearly centered between the two hip bones. This belt design may allow the alignment device to be fully embedded into a slot on the belt, or to have a space along the belt that allows for a replaceable adhesive to stick.

For this type of alignment device, the alignment or misalignment of the pelvis may be tracked, measured and shared with the separate electronic device, the remote server and/or software executed by the alignment device in order to provide actionable feedback to the user.

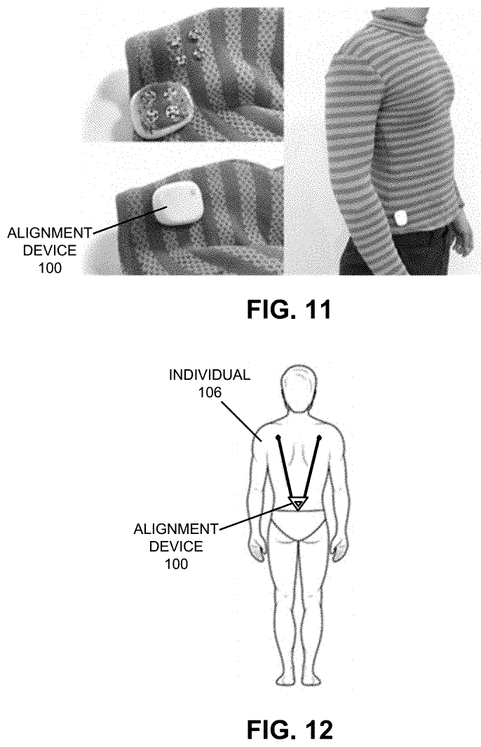

Moreover, in some embodiments, the alignment device may be included in or attached to a part of a user's or person's clothing (e.g., an integrated attachment device). This is shown in FIG. 11, which presents a drawing illustrating front and perspective views of an example of clothing-based attachment of an alignment device.

While FIG. 11 illustrates an example of a type of integration, in general the alignment device may be located on the user's back, such as in or near the center of the two hip bones in order to measure pelvic posture and alignment. Thus, FIG. 11 provides a general illustration of how an electronic device can be integrated into clothing. In general, the type of clothing may include: shirts, pants, jackets, sweatshirts, sportswear, etc.

For this type of alignment device, the alignment or misalignment of the pelvis may be tracked, measured and shared with the separate electronic device, the remote server and/or software executed by the alignment device in order to provide actionable feedback to the user.

In some embodiments, the placement and/or orientation of the alignment device and/or one or more alignment sensors may be based at least in part on a type of activity (such as a sport) and/or a type of complaint of an individual (such as based at least in part on where they experience pain). For example, the alignment device and/or one or more alignment sensors may be placed at appropriate locations and/or orientations based at least in part on a type of activity, such as golf or tennis.

Furthermore, in some embodiments, the alignment device may be located in or near a center of the lower back between the hips, e.g., with two rubber cables going up the back and remateably attached to a fixed position on the scapulas or shoulder blades. This is shown in FIG. 12, which presents a drawing illustrating an example of scapula-based attachment of an alignment device.

As shown in FIG. 12, there may be a cable anchored to each scapula and coupled to the attachment device located around the tailbone. By establishing a line-based reference system, the attachment device may measure tension changes/differences between sides of the body. This approach may allow the attachment device to identify both upper-body posture and pelvic posture, twisting/misalignment, etc.

In some embodiments, the line-based reference system may be attached to the lower scapula as opposed to the mid-to-upper scapula region shown in FIG. 12. However, the line-based reference system would operate in a similar manner.

For this type of alignment device, the alignment or misalignment of the upper body and the pelvis may be tracked, measured and shared with the separate electronic device, the remote server and/or software executed by the alignment(s) device in order to provide actionable feedback to the user. Additionally, this type of alignment-device arrangement may allow upper-body rotation, twisting, and/or misalignment to be tracked.

In some embodiments, an implanted (or in the body) alignment sensor may be used for tracking and identifying misalignment of the pelvis. This implanted alignment sensor may be a semi-passive sensor. For example, a semi-passive sensor may include a radio-frequency identification (RFID) tag with a battery and/or that take energy from the background or the environment for communication.

We now further describe features of the alignment device. The alignment device may, e.g., have a width or length that is at least the diameter of a quarter, and a thickness of less than 1 cm. Moreover, the alignment device may remateably fasten or attach to the body, or may be attached to a belt or another article of clothing. The alignment device may be reusable. Furthermore, the alignment device may include one or more alignment sensors and/or may communicate with one or more separate or external alignment sensors. The alignment device may communicate with a separate electronic device that executes an application. The alignment device may collect data, and may provide suggestions/recommendations (which are sometimes referred to as ‘feedback’) for how a user can correct posture misalignment.

In some embodiments, the posture alignment may be assessed remotely via one or more measurements. For example, the posture alignment may be assessed by an image or a video acquired by a separate imaging device. Alternatively or additionally, the posture alignment may be assessed using pressure sensors on a floor that monitor an individual's gait. Note that this remote alignment monitoring may be used in lieu of or in conjunction with the embodiments of the alignment device described previously.

Moreover, the separate imaging device may be static (such as a stationary imaging device, e.g., a camera, a webcam, etc.) or dynamic (such as an imaging device that follows you, e.g., a drone), and may be used to monitor pelvic posture.

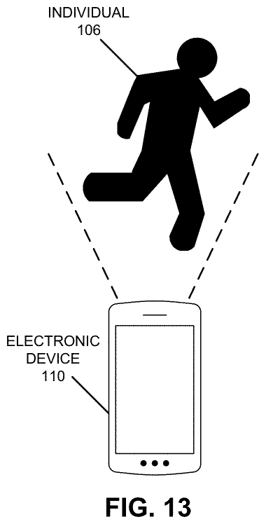

FIG. 13 presents a drawing illustrating an example of posture monitoring using an electronic device that includes an image sensor.

The imaging device may acquire or collect images in a manner that takes into account surface planes, such as the floor, a table/desk, etc. This may allow an individual's pelvic posture to be calculated relative to a surface or plane in the image itself. Additionally, the plane of objects within an image can provide a plane of reference to correctly align an image.

Note that mathematical rotation in combination with a plane of reference can facilitate semi-accurate rotation of an object in an image. For example, the following equations illustrate ways to rotate an image or object in an image given x and y coordinates:

R_{(0,0),90°}(x,y)=(−y,x);

R_{(0,0),180°}(x,y)=(−x,−y); and

R_{(0,0),-90°}(x,y)=(y,−x).

In some embodiments, these equations can be used to analyze an image to identify how a user is standing and/or their pelvic posture.

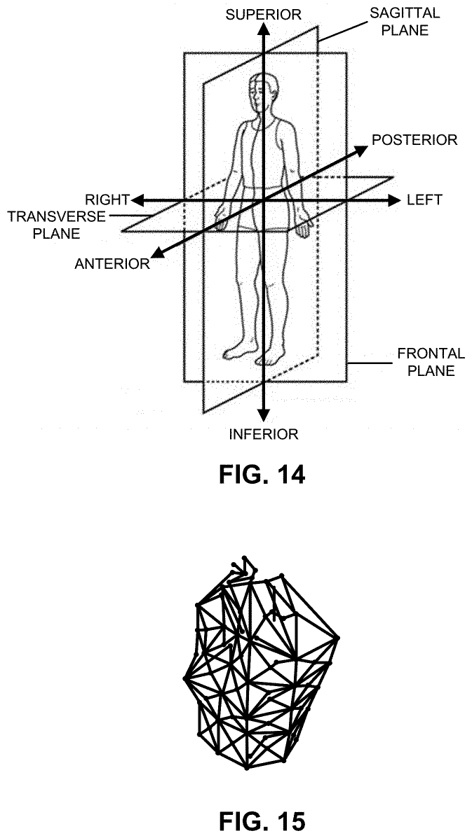

Moreover, in embodiments where the measurements include an image, the analysis of the measurements may be performed using a pretrained neural network, such as a convolutional neural network. Note that the analysis may determine the occurrence of posture misalignment relative to anatomical planes of the body. FIG. 14 presents a drawing illustrating an example of anatomical planes of the human body.

In some embodiments, the alignment sensors associated with the alignment device may have multiple nodes spread across a surface (e.g., embedded in an article of clothing) and that are arranged in a mesh network. This is shown in FIG. 15, which presents a drawing illustrating an example of a mesh network. The network of nodes (which may include the sensors) in the mesh network (which may cover at least a portion of the body, such as the anterior and/or the posterior thorax) can identify misalignments and incorrect pelvic posture, but it may also identify strained/constricted/affected muscles. In the case of misalignment, the mesh network can vibrate or provide tactile feedback to muscles that require hands on relief (e.g., massage). Alternatively, in the case of strained or constricted muscles, the mesh network can sense muscles that are in distress and may provide real-time feedback to the alignment device/end user. Additionally, the use may indicate whether they want the alignment device to provide feedback based at least in part on the measurements/assessment (e.g., tilting of the pelvis, tilting and rotation of the pelvis, etc.).

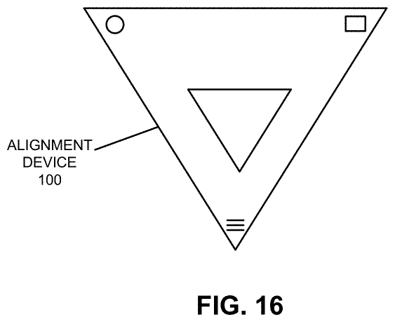

The feedback provided by the alignment system may be unique or specific to the individual and the posture misalignment. For example, there may be a variety of types of feedback, such as: vibration, tactile pressure, sound, light, etc. In some embodiments, the alignment device may have multiple contact points or nodes that are in contact with the body and areas that emit light to tell the user their pelvic posture status (e.g., red, yellow or green). FIG. 16 presents a drawing illustrating an example of an alignment-system feedback structure.

In the preceding discussion, a number of different data collection techniques are described, including a single alignment device (which may be attached on or proximate to an individual's tailbone), a group of sensors (which may be integrated into clothing), etc. In some embodiments, a standardized data model may be used. The alignment device and/or sensor may feed data into the standardized data model, so that different components in the alignment system can interact and/or collaborate with each other. For example, a single sensor on or proximate to the tailbone may be used for when an individual is working (such as on a workday during the week), but multiple sensors may provide more a detailed assessment or measurements when an individual is working out or exercising.

In some embodiments, there can be various types of feedback (such as light, tactile pressure and/or vibration) at different physical positions in the alignment device. These outputs may provide the user with real-time feedback regarding their pelvic posture as measured and determined by the alignment device. Moreover, the outputs may include: light or subtle vibrations, applied pressure, or emitted light that suggest or indicate that the user is out of posture alignment. Alternatively or additionally, the feedback may be directly communicated with an electronic device (such as a cellular telephone) via an application executed on the electronic device.

The real-time communication between the alignment device and the electronic device may trigger alarms, but may also be provide data that can identify particular trends or behaviors about the user's posture. The patterns and user trends relating to their posture can be identified and tracked using machine-learning pattern recognition, such as using a pretrained neural network or a pretrained supervised-learning model (which may be trained using a training dataset and a supervised-learning technique, such as: support vector machines, classification and regression trees, logistic regression, LASSO, logistic LASSO regression, linear regression, a Bayesian technique, and/or another linear or nonlinear supervised-learning technique). For example, the user may habitually lean on one leg, resulting in posture misalignment. This posture misalignment may be monitored and analyzed continuously, periodically or as-needed to identify changes over time. With this information, the alignment device may identify and suggest specific feedback to correct this misalignment, thereby helping the user resolve this pattern of behavior. Additionally, the data collected over time may be correlated or associated with specific activities or behaviors, such as work versus exercise. This information may be monitored according to a daily and/or weekly schedule (e.g., set hours for working, exercise or leisure).

Note that there may be two more alignment-device modes for processing data and providing feedback, including: a monitoring mode and a kinesthetic mode. The following description uses use cases directly associated with the alignment device as illustrations of the feedback techniques.

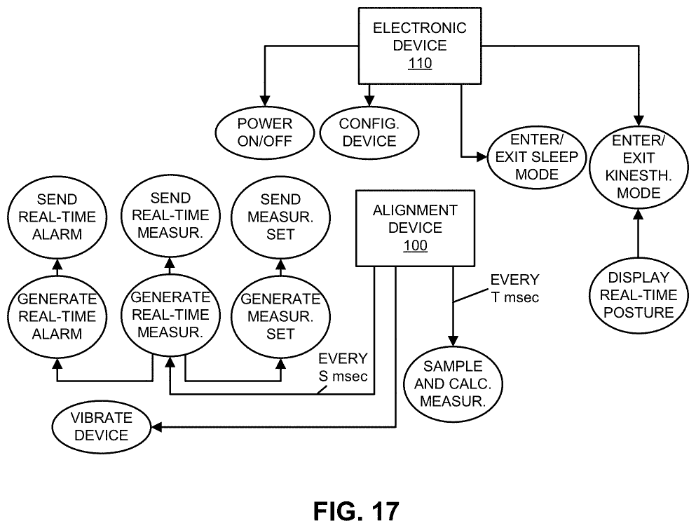

FIG. 17 presents a drawing illustrating an example of an alignment-device interaction in the different alignment device modes. Notably, the user may power on the alignment device to use it and may power off the alignment device when it's not in use. Moreover, the user may configure the alignment device in a number of ways, e.g., via a user interface (such as using a keyboard, a touchpad, a stylus, a mouse, a touch-sensitive display, a voice-recognition engine, etc.) associated with the application executing on the electronic device.

The alignment device may default to (e.g., it may automatically revert to after a time period) the monitoring mode. However, a user may set the alignment device to the kinesthetic mode for real-time posture/alignment feedback.

In addition to the two active modes, the alignment device may determine autonomously that there has been little movement or activity over a time interval (such as when the user is asleep) and that the alignment device should therefore go into sleep mode. The user may also manually designate the sleep mode, e.g., via the user interface associated with the application executed on the electronic device.

Moreover, the alignment device may continuously, periodically or as-needed sample the user alignment and may generate a posture or an alignment measurement. This alignment measurement may be stored and then analyzed in one or more different ways depending on the current mode of the alignment device. When the alignment device is in the kinesthetic mode, then the alignment device may send measurements to the electronic device (such as the user's cellular telephone) at a broadcast rate (e.g., every second). Furthermore, when the alignment device is in the monitoring mode, then the alignment device may compare the measurement to a triggering criterion and, when the triggering criterion is met, may trigger the alignment device to provide feedback (such as by vibrating) and may also send an alarm to the electronic device. Additionally, when the alignment device is in the kinesthetic mode, then the alignment device may store sets of one or more measurements and then may send the sets to the electronic device at a relatively low broadcast rate (e.g., every 10 seconds). Note that when there is no connection to the electronic device, the stored data may be aggregated and then may be sent in a greater volume and at a higher data rate when the connection is established in order to catch up with the missed broadcasts.

The alignment device may be connected to various types of electronic devices, such as mobile/handheld electronic devices or IoT-based devices. For example, the alignment device may be connected to a user's smartphone or tablet. Alternatively, the alignment device may connect to one or more other types of electronic devices, such as a smart speaker or a home assistant. These electronic devices may be capable of fully integrating with the user's alignment device and the remote server.

In some embodiments, the remote or cloud-based server may provide software as a service (SaS). The SaS may include the alignment device sending data to the cloud-based server for analysis and/or receiving feedback from the cloud-based server. This feedback may be based at least in part on real-time pelvic posture data, historical pelvic-posture data for one or more individuals, and may include ways to improve pelvic posture (e.g., muscle massage).

Moreover, in some embodiments, there may be at least two modes of communication. Notably, the alignment device may have different communication modes for data collection and transmission. For example, the alignment device may collect data in a buffer and may transmit the data based at least in part on a request from the remote server. Alternatively or additionally, the alignment device may establish a connection with another IoT device (such as a smart speaker) or another local computing device or electronic device (such as a cellular telephone), and then may upload data to a remote server via the other IoT device and/or the other local computing device. When the alignment device is connected with a mobile electronic device (such as a cellular telephone), this electronic device may be responsible for collecting data from the alignment device for optional local analysis and/or for sending this data to the remote server for at least a portion of the analysis.

Data measurements from the alignment device may be stored in the cloud-based server. Based at least in part on this data, periodic analysis (such as every 10 min. 30 min. hour, 3 hours, 6 hours or daily) may be performed by one or more analysis agents or program instructions that analyze the data and/or convert it into a form suitable for one or more pretrained machine-learning models, such as a pretrained neural network or a pretrained supervised-learning model. The one or more machine-learning models may identify and/or categorize posture deviations based at least in part on the data and may provide one or more recommendations for one or more associated pain or posture management therapies. These recommendations may be made available via one or more push notifications, via the electronic device and/or one or more Web applications, along with one or more comprehensive analytics reports describing the findings. The one or more analytics reports may segregate time intervals characterized by pronounced or exaggerated posture deviations, e.g., during work hours at a standing desk or in the early morning while exercising, to provide therapies or interventions directed or associated with specific activities. Moreover, therapy feedback from customers may be incorporated into the categorization and therapy-selection techniques to revise the recommendations (and, more generally, the feedback).

In addition to the data management and the analysis, the remote server may also manage a user's profile. Note that the data on or associated with the remote server may include: data measured by the alignment device; profile information/settings from a customer (such as settings for how the alignment device is configured, how the application is configured, how a Web application accessed via a Web browser is presented, etc.); derived data measurements that are stored in a format that the one or more pretrained machine-learning models can analyze; one or more recommendations; and/or one or more generated reports for a customer.

From an interaction perspective, the alignment system may use a pseudo ‘push’ notification (such as a push hypertext transfer protocol or HTTP push notification) in which a cellular telephone can check to see if there is a new report, some specific findings that have not yet been reviewed, etc. In some embodiments, the periodicity of reports and modules executing may be on the order of days. However, in these embodiments, the periodicity may be variable or dynamic. Alternatively, in other embodiments, a report may be generated and provided dynamically or in real time. For example, assume that a posture misalignment or deviation is detected during the afternoon of work hours, then the application executing on the electronic device may provide an alert akin to ‘you're doing it again’ based at least in part on predefined or predetermined specific alert ranges. Moreover, the alert may reference or point to a recent report in which the alignment system identified declining posture during afternoon work hours.

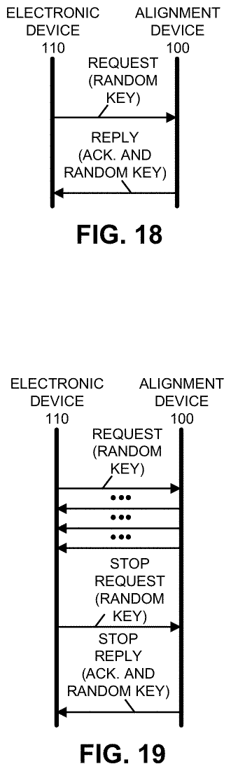

The alignment-device connection technique may include multiple elements related to the alignment device. Notably, there may be two types of alignment-device to electronic-device connection techniques: continuous, and non-continuous. The non-continuous type of connection technique is illustrated in FIG. 18, which presents a drawing illustrating an example of communication between alignment device 100 and electronic device 110.

In either of the connection techniques, electronic device 110 may send a request with a randomly generated key to alignment device 100. In response, alignment device 100 may send back a reply that acknowledges the request and sends back the random key. In the non-continuous type of connection technique, the information is sent based at least in part on a time constraint. Moreover, in the case of the continuous type of connection technique, there may be a start request provided to alignment device 100 from electronic device 110 and multiple replies with the requested information until a stop reply and request is returned to alignment device 100 from electronic device 110. This is illustrated in FIG. 19, which presents a drawing illustrating an example of communication between electronic devices in FIG. 1.

Note that the alignment device may not require calibration based at least in part on its placement. Notably, in some embodiments, the user may be responsible for attaching the alignment device to their body in reference to their alignment. Thus, calibration based at least in part on the alignment-device placement may not be required. Stated differently, the feedback technique executing on the alignment device may use the measured samples/data and the derived measurements from the alignment device, and may not need to apply filtering or adjustment in an attempt to calibrate the alignment device because of its placement. Note that the interactive mode described below may be used to assist a user in alignment-device placement.

As discussed previously, the alignment device may operate in two distinct modes: the monitoring mode, and the kinesthetic mode. In the monitoring mode, the alignment device may calculate measurements (roll, pitch and/or yaw) from samples/data and may securely communicate the measurements to the electronic device using a communication protocol, such as BLE. The data rate for monitoring mode may be relatively slow (such as once per second) and may be a bulk data transfer (e.g., multiple measurement datasets may be transferred in a set of packets or frames).

The alignment device may process the measurements and may determine when an alarm should be triggered. When an alarm occurs, the alignment device may provide feedback (such as a haptic response). In some embodiments, the alarm or the feedback may also be communicated at a near real-time data rate to the electronic device via a separate application programming interface (API), so that the electronic device may also provide feedback.

In the kinesthetic mode, the alignment device may communicate measurements in a near real-time/streaming manner to the electronic device to assist with establishing a connection with the electronic device and to provide a baseline with specific levels of posture alignment.

Note that in the sleep mode, the alignment device may be turned off and de-activated.

The alignment device may perform threshold monitoring in order to activate a trigger when the user maintains a misaligned pelvic posture. This trigger may be based at least in part on particular mean or average values of roll, pitch and/or yaw (or values in another two or three-dimensional coordinate system) relative to predefined or predetermined thresholds and may result in a specific trigger response.

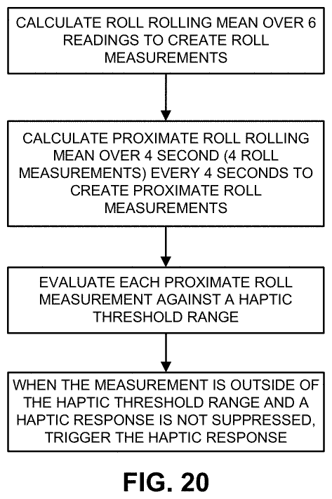

FIG. 20 presents a flow diagram illustrating an example method for alignment triggering using an electronic device in FIG. 1. Notably, as shown in FIG. 20, the method may start with the alignment device calculating mean or average values of the roll over, e.g., six readings to create roll measurements. Then, the alignment device may calculate mean or average values of roll over four seconds (e.g., four roll measurements) every four seconds to create a set of proximate roll measurements. Each of the proximate roll measurements may be evaluated/compared to a haptic threshold range. When the measurement is outside of the threshold range and haptics are not suppressed, a haptic response or feedback may be triggered.

The triggering technique may take into account near-term historical data and/or recent provided triggers, and may incorporate a backoff technique or hysteresis to ensure that the alignment device is not constantly triggering even when the user is out of alignment beyond established thresholds.

The feedback technique may be parameterized as much as possible to allow for post-manufacture alignment-technique adjustments, as well as user customization. Defaults may be available locally to the alignment device, but can be overridden using the user interface associated with the application executed on the electronic device.

We now describe the analysis of the measurements. Notably, the following discussion provides information regarding embodiments of a program instructions, elements or modules used in the analysis of the measurements.

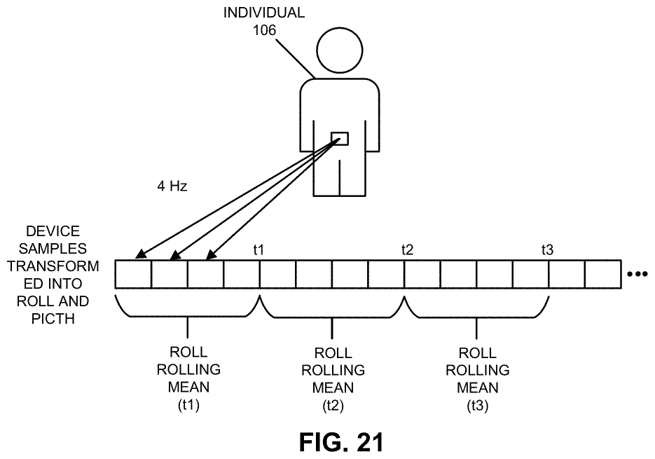

The analysis by the alignment device may start with sampling. For example, the alignment device may take or acquire a sample every 250 milliseconds (four times/second) and from a given sample may calculate values of roll, pitch and/or yaw (which are sometimes referred to as ‘measurements’). 4-16 consecutive measurements may be used to compute a rolling average or mean value, which may be calculated every 1000 milliseconds (1 time/second). This is shown in FIG. 21, which presents a drawing illustrating an example of sampled measurements.

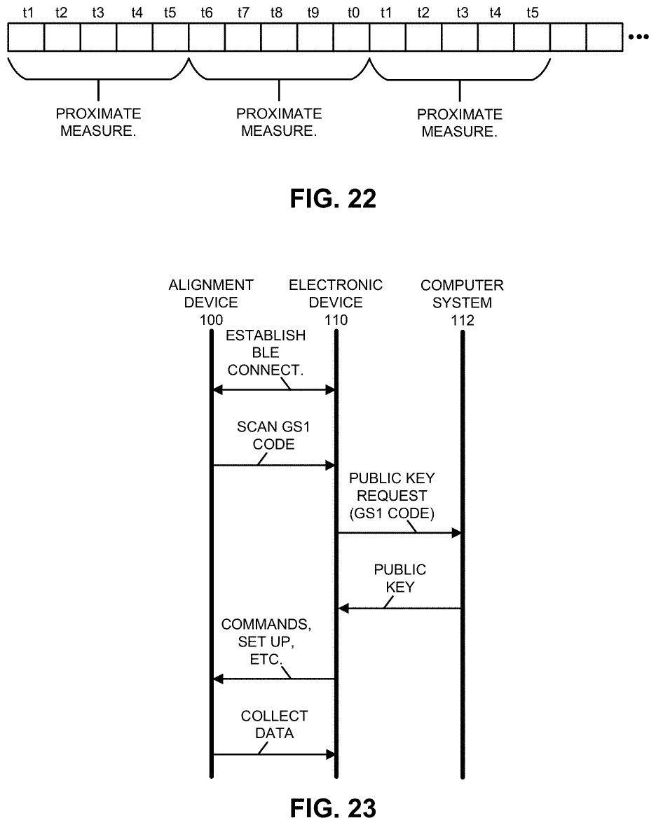

The average or mean for roll, pitch and/or yaw over a 1-10 s time interval (which is sometimes referred to as ‘proximate monitoring’) may be used to trigger an alarm. The triggering rate may be long enough to avoid being annoying, but may also be low or frequent enough to provide effective feedback to the individual. This duration may be configurable, both in terms of a default value and a user-specific value. For example, a trigger may occur every 5, 15, 30 or 60 s. FIG. 22 presents a drawing illustrating an example of proximate measurements of 5 s.

Not that feedback, such as a haptic trigger, may be engaged when the proximate roll measurement is outside a predefined or predetermined haptic threshold range. For example, the initial feedback technique may use a roll haptic threshold range of −7.5° to 7.5°. When the number of triggers within a monitoring time interval exceeds a threshold (such as 2-5 per minute), then trigger suppression may occur for a backoff time (such as several minutes) to avoid excessive triggering. Haptic triggering may also be provided via an optional alarm that is sent to the electronic device.

As discussed previously, communication with the electronic device may be performed via BLE. For example, there may be alignment-device operational and state configuration communication from the user's cellular telephone to the alignment device. For example, general configuration information may be provided to define specific system operational parameters. Note that at least some of the system operational parameters may be configured by the user, while others may be automatically determined after use of the alignment device. Moreover, several different modes (such as the monitoring mode, the kinesthetic mode or the sleep mode) may be set using the cellular telephone. The default state may be the monitoring mode and, as discussed previously, the user may exit the monitoring mode and enter and/or then exit the kinesthetic mode or the sleep mode. The sleep mode may be used when turning off the alignment device when it's not in use. Alternatively, the alignment device may put itself into sleep mode when it does not detect any movement of the individual, e.g., for several minutes.

BLE communication from alignment device to the electronic device may upload or provide measurement and alarm data. When in the monitoring mode, measurement data may be uploaded, e.g., every 10 s. However, when the electronic device is not connected or able to communicate with the alignment device, then data may be stored in the alignment device until the connection is established, thereby allowing the electronic device to catch up. In some embodiments, alarm data may be uploaded as soon as it is detected/measured. When in the kinesthetic mode, the measurement upload rate may be smaller (e.g., every one second), which may allow the electronic device to display near real-time posture values.

Table 1 presents pseudo-code corresponding to the analysis of the measurements. Note that the pseudo-code is supported by comment blocks to provide context. In some embodiments, the content (such as the measurements, analysis results, the feedback, etc.) may be passed through one or more APIs and may have a format that is compatible with JavaScript Object Notation (JSON).

```
TABLE 1
Connect alignment device
Configure the alignment device
Set/change mode
Sample and calculate measurement
  final static double SAMPLE_RATE_IN_SECONDS = 1.0/4.0;
  final static double
  ROLLING_MEAN_DURATION_IN_SECONDS = 1.0;
  final static integer
  NUMBER_OF_MEASUREMENTS_IN_PROXIMATE_{—}
    MEASUREMENT = 15;
  final static double
  PROXIMATE_MEASUREMENT_DURATION_IN_{—}
    SECONDS = ROLLING_MEAN_DURATION_IN_SECONDS•
    NUMBER_OF_MEASUREMENTS_IN_PROXIMATE_{—}
    MEASUREMENT;
  every (SAMPLE_RATE) {
    // Record current timestamp
    // Sample 3-axis gyroscope, 3-axis accelerometer and 3-axis compass
      // Generate measurements: roll, pitch and yaw
        // Store roll, pitch and yaw with timestamp
  }
  every (ROLLING_MEAN_DURATION_IN_SECONDS) {
    // Calculate rolling mean for roll, pitch and yaw for
    // current rolling mean timestamp
    // Store rolling mean for roll, pitch and yaw with
    // rolling mean timestamp
  }
  every
  (PROXIMATE_MEASUREMENT_DURATION_IN_SECONDS) {
    // Calculate proximate roll, pitch and yaw measurements for
      // current proximate measurement timestamp
      // Store proximate roll, pitch and yaw measurement with
      // proximate measurement timestamp
  }
Generate and send measurement set
Generate and send real-time measurement
Generate and send real-time alarm
```

We now describe security and encryption protocols. The electronic device may be a client in a client-server wireless connection. Regardless of the wireless protocol being used, the electronic device may enforce or maintain a single attachment to a remote server. For example, when the communication protocol used is BLE, the electronic device may be attached to a single remote server and may be detached before it can be connected to a different server or computer.

After a connection is established, the electronic device may not allow communication that is not encrypted (e.g., using an asymmetric or a symmetric encryption protocol) and approved by both the electronic device and the remote server. Note that each electronic device and/or each alignment device may retain a unique identity that is known only to the remote server and to the electronic device.

Upon connecting the alignment device to the remote server (e.g., via the electronic device), the electronic device may expect only encrypted messages that it can read will be sent to it. Otherwise, the electronic device may not respond. As noted previously, each electronic device and/or each alignment device may have a unique code or identifier, which is stored on the electronic device and/or the alignment device. This code may be used as a unique identity for the electronic device and/or the alignment device. For example, the unique code may be a GS1 barcode (of Brussels, Belgium), which can be scanned by a cellular telephone or the electronic device. With this unique code, the application executing on the electronic device may retrieve the public key of the alignment device (e.g., from the remote server) and may use this public key to encrypt messages that are communicated in the alignment system.

In some embodiments, the alignment device and the application executing on the electronic device may establish new non-permanent (per-session) encryption using both of their relative public keys via, e.g., a Diffie—Hellman (DH) technique. Note that the DH technique may include a key-exchange protocol that enables two parties communicating over a public channel to establish a mutual secret without it being transmitted over a network. The DH technique may enable the two parties to use a public key to encrypt and decrypt their communications or data using symmetric cryptography. However, in general, a wide variety of encryption techniques may be used.

FIG. 23 presents a drawing illustrating an example of communication between alignment device 100, electronic device 110 and computer system 112 (which may include or may perform the operations of the server).

As noted previously, the DH technique may be used to establish a secure session between the application executing on the electronic device and the remote server. Messages communicated over a connection may be encrypted using the DH technique. Moreover, the unique identity of the application may be established during setup of the electronic device, which may also define public and private keys. The electronic device may use its public key to establish a secure connection with the remote server.

We now describe embodiments of an electronic device, which may perform at least some of the operations in the feedback techniques. Note that the electronic device may include alignment device 100, electronic device 110, a computer in computer system 112, one of access points 116 and/or one of radio nodes 118. FIG. 24 presents a block diagram illustrating an example of an electronic device 2400 in accordance with some embodiments. This electronic device includes processing subsystem 2410, memory subsystem 2412, and networking subsystem 2414. Processing subsystem 2410 includes one or more devices configured to perform computational operations. For example, processing subsystem 2410 can include one or more microprocessors, ASICs, microcontrollers, programmable-logic devices, one or more graphics process units (GPUs) and/or one or more digital signal processors (DSPs).

Memory subsystem 2412 includes one or more devices for storing data and/or instructions for processing subsystem 2410 and networking subsystem 2414. For example, memory subsystem 2412 can include dynamic random access memory (DRAM), static random access memory (SRAM), and/or other types of memory. In some embodiments, instructions for processing subsystem 2410 in memory subsystem 2412 include: one or more program modules or sets of instructions (such as program instructions 2422 or operating system 2424), which may be executed by processing subsystem 2410. Note that the one or more computer programs may constitute a computer-program mechanism. Moreover, instructions in the various modules in memory subsystem 2412 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 2410.

In addition, memory subsystem 2412 can include mechanisms for controlling access to the memory. In some embodiments, memory subsystem 2412 includes a memory hierarchy that comprises one or more caches coupled to a memory in electronic device 2400. In some of these embodiments, one or more of the caches is located in processing subsystem 2410.

In some embodiments, memory subsystem 2412 is coupled to one or more high-capacity mass-storage devices (not shown). For example, memory subsystem 2412 can be coupled to a magnetic or optical drive, a solid-state drive, or another type of mass-storage device. In these embodiments, memory subsystem 2412 can be used by electronic device 2400 as fast-access storage for often-used data, while the mass-storage device is used to store less frequently used data.

Networking subsystem 2414 includes one or more devices configured to couple to and communicate on a wired and/or wireless network (i.e., to perform network operations), including: control logic 2416, an interface circuit 2418 and one or more antennas 2420 (or antenna elements) and/or input/output (I/O) port 2430. (While FIG. 24 includes one or more antennas 2420, in some embodiments electronic device 2400 includes one or more nodes, such as nodes 2408, e.g., a network node that can be coupled or connected to a network or link, or an antenna node or a pad that can be coupled to the one or more antennas 2420. Thus, electronic device 2400 may or may not include the one or more antennas 2420.) For example, networking subsystem 2414 can include a Bluetooth™ networking system (such as Bluetooth classic or BLE), a cellular networking system (e.g., a 3G/4G/5G network such as UMTS, LTE, etc.), a universal serial bus (USB) networking system, a networking system based on the standards described in IEEE 802.11 (e.g., a Wi-Fi® networking system), an Ethernet networking system, a cable modem networking system, and/or another networking system.

Networking subsystem 2414 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, electronic device 2400 may use the mechanisms in networking subsystem 2414 for performing simple wireless communication between the electronic devices, e.g., transmitting advertising or beacon frames and/or scanning for advertising frames transmitted by other electronic devices.

Within electronic device 2400, processing subsystem 2410, memory subsystem 2412, and networking subsystem 2414 are coupled together using bus 2428. Bus 2428 may include an electrical, optical, and/or electro-optical connection that the subsystems can use to communicate commands and data among one another. Although only one bus 2428 is shown for clarity, different embodiments can include a different number or configuration of electrical, optical, and/or electro-optical connections among the subsystems.

In some embodiments, electronic device 2400 includes a display subsystem 2426 for displaying information on a display, which may include a display driver and the display, such as a liquid-crystal display, a multi-touch touchscreen, etc.

Electronic device 2400 can be (or can be included in) any electronic device with at least one network interface. For example, electronic device 2400 can be (or can be included in): a hair-styling tool, a computer, a computer system, a desktop computer, a laptop computer, a subnotebook/netbook, a tablet computer, a smartphone, a cellular telephone, a smartwatch, a consumer-electronic device, a portable computing device, and/or another electronic device.

Although specific components are used to describe electronic device 2400, in alternative embodiments, different components and/or subsystems may be present in electronic device 2400. For example, electronic device 2400 may include one or more additional processing subsystems, memory subsystems, networking subsystems, and/or display subsystems. Additionally, one or more of the subsystems may not be present in electronic device 2400. Moreover, in some embodiments, electronic device 2400 may include one or more additional subsystems that are not shown in FIG. 24, such as a user-interface subsystem 2432. Also, although separate subsystems are shown in FIG. 24, in some embodiments some or all of a given subsystem or component can be integrated into one or more of the other subsystems or component(s) in electronic device 2400. For example, in some embodiments program instructions 2422 are included in operating system 2424 and/or control logic 2416 is included in interface circuit 2418.

Moreover, the circuits and components in electronic device 2400 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit (which is sometimes referred to as a ‘communication circuit’) may implement some or all of the functionality of networking subsystem 2414 (or, more generally, of electronic device 2400). The integrated circuit may include hardware and/or software mechanisms that are used for transmitting wireless signals from electronic device 2400 and receiving signals at electronic device 2400 from other electronic devices. Aside from the mechanisms herein described, radios are generally known in the art and hence are not described in detail. In general, networking subsystem 2414 and/or the integrated circuit can include any number of radios. Note that the radios in multiple-radio embodiments function in a similar way to the described single-radio embodiments.

In some embodiments, networking subsystem 2414 and/or the integrated circuit include a configuration mechanism (such as one or more hardware and/or software mechanisms) that configures the radio(s) to transmit and/or receive on a given communication channel (e.g., a given carrier frequency). For example, in some embodiments, the configuration mechanism can be used to switch the radio from monitoring and/or transmitting on a given communication channel to monitoring and/or transmitting on a different communication channel. (Note that ‘monitoring’ as used herein comprises receiving signals from other electronic devices and possibly performing one or more processing operations on the received signals)

In some embodiments, an output of a process for designing the integrated circuit, or a portion of the integrated circuit, which includes one or more of the circuits described herein may be a computer-readable medium such as, for example, a magnetic tape or an optical or magnetic disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While the preceding discussion used particular communication protocols as an illustrative example, in other embodiments a wide variety of communication protocols and, more generally, wired and/or wireless communication techniques may be used. Thus, the feedback techniques may be used in conjunction with a variety of network interfaces. Furthermore, while some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the feedback techniques may be implemented using program instructions 2422, operating system 2424 (such as a driver for interface circuit 2418) or in firmware in interface circuit 2418. Alternatively or additionally, at least some of the operations in the feedback techniques may be implemented in a physical layer, such as hardware in interface circuit 2418.

In the preceding description, we refer to ‘some embodiments.’ Note that ‘some embodiments’ describes a subset of all of the possible embodiments, but does not always specify the same subset of embodiments. Moreover, note that numerical values in the preceding embodiments are illustrative examples of some embodiments. In other embodiments of the feedback techniques, different numerical values may be used.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. An alignment device, comprising:
an integrated circuit configured to:
obtain measurements associated with a pelvic posture of an individual;
analyze the measurements to determine when a posture misalignment occurs; and
when the posture misalignment occurs, provide feedback intended for the individual, wherein the alignment device is configured to remateably attached or coupled to the individual.

2. The alignment device of claim 1, wherein the alignment device is remateably attached or coupled to the individual near a midline of the posterior lumbar or sacral region.

3. The alignment device of claim 1, wherein the alignment device comprises one or more alignment sensors configured to perform the measurements; and
wherein obtaining the measurements comprises acquiring the measurements using the one or more alignment sensors.

4. The alignment device of claim 3, wherein the one or more sensors comprise: an orientation sensor, a gyroscope, a compass, or an accelerometer.

5. The alignment device of claim 1, wherein the alignment device comprises an interface circuit configured to communicate with one or more alignment sensors that are external to the alignment device; and
wherein obtaining the measurements comprises acquiring the measurements using the one or more alignment sensors.

6. The alignment device of claim 1, wherein the alignment device comprises an interface circuit configured to communicate with an electronic device; and
wherein analyzing the measurements comprises:
providing, addressed to the electronic device, the measurements; and
receiving, associated with the electronic device, results of the analysis, instructions for the feedback, or both.

7. The alignment device of claim 1, wherein the feedback comprises: an indication that the posture misalignment has occurred; or a recommended therapy to correct the posture misalignment.

8. The alignment device of claim 1, wherein the feedback comprises a sensory output intended for the individual.

9. The alignment device of claim 1, wherein the feedback comprises: light, sound, or haptic information.

10. The alignment device of claim 1, wherein the posture misalignment comprises a pelvic misalignment.

11. A non-transitory computer-readable medium for use in conjunction with an alignment device, the computer-readable storage medium storing program instructions that, when executed by the alignment device, cause the alignment device to perform operations comprising:
obtaining measurements associated with a pelvic posture of an individual;
analyzing the measurements to determine when a posture misalignment occurs; and
when the posture misalignment occurs, providing feedback intended for the individual, wherein the alignment device is remateably attached or coupled to the individual.

12. The non-transitory computer-readable medium of claim 11, wherein the alignment device is remateably attached or coupled to the individual near a midline of the posterior lumbar or sacral region.

13. The non-transitory computer-readable medium of claim 11, wherein obtaining the measurements comprises acquiring the measurements using the one or more alignment sensors in the alignment device.

14. The non-transitory computer-readable medium of claim 11, wherein obtaining the measurements comprises acquiring the measurements using the one or more alignment sensors that are external to the alignment device.

15. The non-transitory computer-readable medium of claim 11, wherein analyzing the measurements comprises:
providing, addressed to an electronic device, the measurements; and
receiving, associated with the electronic device, results of the analysis, instructions for the feedback, or both.

16. A method for providing feedback, comprising:
by an alignment device:
obtaining measurements associated with a pelvic posture of an individual;
analyzing the measurements to determine when a posture misalignment occurs; and
when the posture misalignment occurs, providing the feedback intended for the individual, wherein the alignment device is remateably attached or coupled to the individual.

17. The method of claim 16, wherein the alignment device is remateably attached or coupled to the individual near a midline of the posterior lumbar or sacral region.

18. The method of claim 16, wherein obtaining the measurements comprises acquiring the measurements using the one or more alignment sensors in the alignment device.

19. The method of claim 16, wherein obtaining the measurements comprises acquiring the measurements using the one or more alignment sensors that are external to the alignment device.

20. The method of claim 16, wherein analyzing the measurements comprises:
providing, addressed to an electronic device, the measurements; and
receiving, associated with the electronic device, results of the analysis, instructions for the feedback, or both.
